(19) United States
(12) Patent Application Publication
(10) Pub. No.: US 2021/0371364 A1
(43) Pub. Date: Dec. 2, 2021

(54) PROCESS FOR THE PREPARATION OF 3,3,5-TRIMETHYLCYCLOHEXYLIDENE BISPHENOL (BP-TMC)

(71) Applicant: Covestro Intellectual Property GmbH & Co KG, Leverkusen (DE)

(72) Inventors: Kristof HEYLEN, Zemst (BE); Johan VANDEN EYNDE, Zwijnaarde (BE); Erik SLUYTS, Brasschaat (BE); Konstantinos METAXAS, Shanghai (CN); Franz BEGGEL, Köln (DE); Michael TRAVING, Burscheid (DE)

(21) Appl. No.: 17/285,344

(22) PCT Filed: Oct. 4, 2019
(86) PCT No.: PCT/EP2019/076926

§ 371 Date: Apr. 14, 2021

(30) Foreign Application Priority Data
Oct. 19, 2018 (EP) 18201372.2

(51) Int. Cl. C07C 37/20; C07C 37/74

(52) U.S. Cl. CPC C07C 37/20; C07C 2601/16; C07C 2601/14; C07C 37/74

(57) ABSTRACT

The present inventions relates to the preparation of 3,3,5-trimethylcyclohexylidene bisphenol. Especially, the present invention relates to the preparation of 3,3,5-trimethylcyclohexylidene bisphenol from 3,3,5-trimethylcyclohexanone and phenol in the presence of a gaseous acidic catalyst. The preparation is preferably conducted continuously.

### CROSS REFERENCE TO RELATED APPLICATIONS

This application is a U.S. national stage application, filed under 35 U.S.C. § 371, of International Application No. PCT/EP2019/076926, which was filed on Oct. 4, 2019, and which claims priority to European Patent Application No. 18201372.2, which was filed on Oct. 19, 2018. The contents of each are incorporated by reference into this specification.

### FIELD

The present inventions relates to the preparation of 3,3,5-trimethylcyclohexylidene bisphenol. Especially, the present invention relates to the preparation of 3,3,5-trimethylcyclohexylidene bisphenol from 3,3,5-trimethylcyclohexanone and phenol in the presence of a gaseous acidic catalyst. The preparation is preferably conducted continuously.

### BACKGROUND

The preparation of 3,3,5-trimethylcyclohexylidene bisphenol, hereinafter also referred to as BP-TMC, from 3,3,5-trimethylcyclohexanone, hereinafter referred to as TMC-one, as a first reactant and phenol as a second reactant in a reaction vessel in the presence of a gaseous acidic catalyst is known per se.

Basically the reaction proceeds as follows according to Scheme 1:

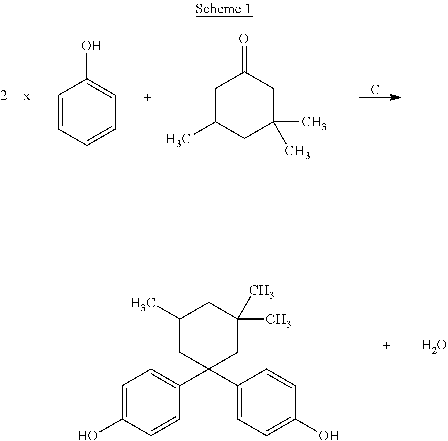

EP0995737A1 discloses the preparation of BP-TMC from TMC-one and phenol in the presence of acidic catalyst already. EP0995737A1 also deals with increasing the yield of TMC-one obtained from the reaction of BP-TMC from TMC-one and phenol. In this regard EP0995737A1 proposes to allow phenol and TMC-one to react in a prereaction until at least 90 mol % of the ketone has reacted and then to add a further quantity of phenol and/or aromatic hydrocarbon to the reaction mixture in a postreaction. Further, EP0995737A1 discloses that the formation of by-products reduces the yield of TMC-one.

EP1277723A1 also discloses the preparation of bisphenols from ketones and phenol in the presence of acidic catalyst already, too. The acidic catalyst may be a mixture of gaseous hydrogen chloride and hydrogen sulfide for example. Further, EP1277723A1 also deals deals with increasing the yield of bisphenol obtained from the reaction of a ketone, e.g. 3,3,5-trimethylcyclohexanone, and phenol. In this regard EP1277723A1 proposes to slow down or stop the reaction speed by addition of water. Furthermore, EP1277723A1 teaches to separate by-products and reaction components as completely as possible from the bisphenol.

The content of EP1277723A1 is incorporated into the present description by reference.

According EP1277723A1 the maximum selectivity in EP1277723A1) in regard to bisphenol A (2,2-bis-(4-hydroxyphenyl)-propane (BPA)) which can be achieved is 95.5%. However, EP1277723A1 discloses such a high selectivity for a discontinuous process only.

### SUMMARY

In regard to BP-TMC, high yields are desired, too. Due to the different reaction kinetics in comparison to the production of BPA and the higher tendency of BP-TMC to degrade, it is harder to achieve such high yields. Especially, it is desired to achieve a yield of the formation of BP-TMC from TMC-one and phenol of at least 90%, preferably of at least 95%, more preferably of at least 98%, most preferably of at least 99%, based on the initial amount of TMC-one.

Additionally, a continuous process for the production of BP-TMC is desired.

Thus, it is an object of the present invention to achieve a yield of the formation of BP-TMC from TMC-one and phenol of at least 90%, preferably of at least 95%, more preferably of at least 98%, most preferably of at least 99%, based on the initial amount of TMC-one.

A further object of the present invention is that yields of formation shall be achieved with a continuous process preferably.

Surprisingly, the object of the invention is achieved by the subject matter of claim 1. Preferred embodiments are described in the subsequent claims.

Especially the object of the invention is achieved by a process for preparing 3,3,5-trimethylcyclohexylidene bisphenol (BP-TMC) from 3,3,5-trimethylcyclohexanone (TMC-one) and phenol comprising at least the following steps:
(a) providing an initial mixture comprising an initial amount of TMC-one and an initial amount of phenol and reacting this initial mixture in the presence of an amount of a gaseous acidic catalyst,
(b) obtaining a reaction mixture comprising BP-TMC, phenol, TMC-one, dissolved acidic catalyst, originated from the gaseous acidic catalyst now being dissolved in this product stream, water, and by-products, the phenol being unreacted phenol and the TMC-one being unreacted TMC-one,
(c) removing dissolved acidic catalyst and water from the obtained reaction mixture,
(d) separating a larger amount, preferably an amount of 70 to 95 wt.-%, more preferably an amount of 80 to 90 wt.-%, of the obtained BP-TMC from the unreacted phenol, from the unreacted TMC-one, and from the by-products,
and then either
(e1) returning a larger amount, preferably an amount of 90 to 99.9 wt. %, more preferably an amount of 95 to 99 wt.-%, of the remaining reaction mixture of (d) containing BP-TMC, the unreacted phenol, the unreacted TMC-one, and the by-products obtained in step (d) to step (a), and
(f) removing a smaller amount, preferably an amount of 10 to 0.1 wt. %, more preferably an amount of 5 to 1 wt.-%, of the remaining reaction mixture of (d) containing BP-TMC, the unreacted phenol, the unreacted TMC-one, and the by-products obtained in step (d) towards waste recovery and waste removal,
or
(e2) returning a smaller amount, preferably an amount of 50 to 90 wt.-%, more preferably an amount of 60 to 80 wt.-%, of the obtained BP-TMC, the unreacted phenol, the unreacted TMC-one, and the by-products obtained in step (d) to step (a),
or
(e3) removing a smaller amount, preferably an amount of 50 to 90 wt.-%, more preferably an amount of 60 to 80 wt.-%, of the obtained BP-TMC, the unreacted phenol, the unreacted TMC-one, and the by-products obtained in step (d) towards waste recovery and waste removal.

This larger amount of the obtained BP-TMC, which in step (d) is separated from the unreacted phenol, from the unreacted TMC-one, and from the by-products, is about 10 to 50% wt.-%, preferably 20-40% wt.-%, of the sum of the amounts of the obtained BP-TMC, the unreacted phenol, the unreacted TMC-one, and the by-products.

### DETAILED DESCRIPTION

By-products are isomers of BP-TMC, e.g. The initial mixture and consequently the reaction mixture comprise inevitable impurities also. These inevitable impurities are introduced by the reactants and catalysts, e.g. One skilled in the art knows the types and amounts of all major inevitable impurities. By-products are not inevitable impurities in the meaning of the present invention.

Surprisingly it was found that a yield of the formation of the BP-TMC from TMC-one and phenol of at least 90%, preferably of at least 95%, more preferably of at least 98%, most preferably at least 99% was achieved, based on the initial amount of TMC-one.

The process may be conducted continuously or discontinuously; preferably, the process is conducted continuously.

Further preferably, the reaction between TMC-one and phenol is conducted in a reaction vessel, preferably in a stirred tank reactor or in a loop flow reactor, especially in a stirred tank reactor.

Further surprisingly, it was found that in a continuously conducted process the yield of the formation of the BP-TMC from TMC-one and phenol was the greater the more often step (e1) or (e2) was conducted. This means after conducting the process according to the invention including either the step (e1) or the step (e2) twice—i.e. two cycles of the process—the yield of the formation of the BP-TMC from TMC-one and phenol was the greater than after having conducted the process according to the invention including either the step (e1) or the step (e2) only once—i.e. one cycle of the process only. Furthermore surprisingly, after conducting the process according to the invention including either the step (e1) or the step (e2) three times—i.e. three cycles of the process—the yield of the formation of the BP-TMC from TMC-one and phenol was the greater than after having conducted the process according to the invention including either the step (e1) or the step (e2) only twice—i.e. two cycles of the process only. So, for example, it was found that the yield of the formation of the BP-TMC from TMC-one and phenol was about 82% after one cycle, about 96% after two cycles and more than 99% after three cycles.

These results were not expected since one skilled in the art would not expect the yield would increase since one skilled to the art would assume—without wishing to being bound to a theory—that especially during step (c) obtained BP-TMC would be degraded or removed during the removal of dissolved acidic catalyst and water from the obtained reaction mixture. So, the rate of increase is surprising especially. In the meaning of the present invention one cycle of the process is run through when a volume corresponding to the volume of the initial mixture in the reaction vessel in which at least the steps (a) and (b) are conducted is run through the reaction vessel.

Further preferably, in step (c) dissolved acidic catalyst and water are removed from the reaction mixture by distillation, preferably using a distillation column having a bottom, preferably the bottom temperate being 130° C. at most, more preferably the bottom temperature being from 120° C. to 125° C.

Further preferably, in step (d) the larger amount, preferably an amount of 70 to 95 wt.-%, more preferably an amount of 80 to 90 wt.-%, of the obtained BP-TMC is separated from the unreacted phenol, from the unreacted TMC-one, and from the by-products by at least the following steps:
(d1) converting the obtained BP-TMC into a BP-TMC-phenol-adduct by crystallization,
(d2) separating the BP-TMC-phenol-adduct and filtration.

Further preferably, in a step (d3) the obtained BP-TMC-phenol-adduct is purified by recrystallization in phenol towards at least 99.9 wt.-% purity.

Further preferably, in a step (d4) BP-TMC is obtained from the separated BP-TMC-phenol-adduct by the removal of phenol from the BP-TMC-phenol-adduct, preferably by drying.

Further preferably, the initial amount of TMC-one comprises an amount of freshly added TMC-one and an amount of unreacted TMC-one and wherein the molar ratio between the amount of freshly added TMC-one and the amount of unreacted TMC-one is from 2:° 1 to 15:1, preferably from 3:1 to 12:1, more preferably from 5:1 to 10:1.

Further preferably, the initial amount of phenol comprises an amount of freshly added phenol and an amount of unreacted phenol and wherein the molar ratio between the amount of freshly added phenol and the amount of unreacted phenol is 1:3 or less, preferably from 1:7 to 1:4.

Further preferably, in step (a) the initial mixture comprises 5 to 25 wt.-%, preferably from 10 to 15 wt.-%, by-products, especially when the process according to the invention comprises the process steps (e1) and (f) or (e2) but not the process step (e3). Alternatively preferably, in step (a) the initial mixture comprises or from 1 to 4 wt.-%, preferably from 2 to 3 wt.-% of by-products, especially when the process according to the invention comprises the process steps (e3) but not the process steps (e1) and (f) or (e2).

Further preferably, no water is added is added to process according to the present invention.

Further preferably, the gaseous acidic catalyst contains hydrogen chloride and hydrogen sulfide.

Further preferably, the reaction temperature in the reaction vessel is at least 30° C. and at most 40° C., preferably at least 33° C. and at most 37° C. Preferably, the pressure in the reaction vessel is at least 1 bar absolute and at most 10 bar absolute, preferably at least 1 bar absolute and at most 5 bar absolute, most preferably at least 1 bar absolute and at most 2 bar absolute.

Preferably, the reaction is conducted under three-phase conditions. This means, that there are solid, liquid and gaseous components in the reaction vessel simultaneously. These components are the reactants TMC-one and phenol, the catalyst, the product BP-TMC, water and by-products. Further, there may be inevitable impurities, as explained above. The formed BP-TMC is present in the solid state in the form of crystals of a BP-TMC-phenol-adduct mainly, i.e. more than 90 wt.-%, preferably more than 95 wt.-%, of the obtained BP-TMC; a minor part of the formed BP-TMC is dissolved in phenol, i.e. less than 10 wt.-% preferably less than 5 wt.-%, of the obtained BP-TMC.

Further preferably, the BP-TMC is obtained with a purity of greater 95 wt.-%, preferably of greater 98 wt.-%, more preferably of greater 99 wt.-%, most preferably greater 99.9 wt.-%.

Further preferably, in step (b) the obtained reaction mixture comprises 55 to 70 wt.-% phenol, less than 5 wt. % TMC-one, from 15 to 22 wt.-% BP-TMC, from 3.5 to 5.5 wt.-% dissolved acidic catalyst, from 0.5 to 2 wt.-%, preferably about 1 wt.-% water, and 5 to 20 wt.-% of by-products, wherein the sum of the amounts of unreacted phenol, unreacted TMC-one, BP-TMC, water, and by-products is 100 wt.-%.

The obtained BP-TMC can be used in the production of polycarbonates e.g., especially in the phase boundary process or the melt transesterification process.

1. A process for preparing 3,3,5-trimethylcyclohexylidene bisphenol from 3,3,5-trimethylcyclohexanone and phenol comprising at least the following steps:
(a) providing an initial mixture comprising an initial amount of 3,3,5-trimethylcyclohexanone and an initial amount of phenol and reacting this initial mixture in the presence of an amount of a gaseous acidic catalyst,
(b) obtaining a reaction mixture comprising 3,3,5-trimethylcyclohexylidene bisphenol, phenol, 3,3,5-trimethylcyclohexanone, gaseous acidic catalyst, water, and by-products, the phenol being unreacted phenol and the 3,3,5-trimethylcyclohexanone being unreacted 3,3,5-trimethylcyclohexanone,
(c) removing gaseous acidic catalyst and water from the obtained reaction mixture,
(d) separating a larger amount of the obtained 3,3,5-trimethylcyclohexylidene bisphenol from the unreacted phenol, from the unreacted 3,3,5-trimethylcyclohexanone, and from the by-products,
and then either
(e1) returning a larger amount of the remaining reaction mixture of (d) containing 3,3,5-trimethylcyclohexylidene bisphenol, the unreacted phenol, the unreacted 3,3,5-trimethylcyclohexanone, and the by-products obtained in step (d) to step (a), and
(f) removing a smaller amount of the remaining reaction mixture of (d) containing 3,3,5-trimethylcyclohexylidene bisphenol, the unreacted phenol, the unreacted 3,3,5-trimethylcyclohexanone, and the by-products obtained in step (d) towards waste recovery and waste removal,
or
(e2) returning a smaller amount of the obtained 3,3,5-trimethylcyclohexylidene bisphenol, the unreacted phenol, the unreacted 3,3,5-trimethylcyclohexanone, and the by-products obtained in step (d) to step (a)
or
(e3) removing a smaller amount of the obtained 3,3,5-trimethylcyclohexylidene bisphenol, the unreacted phenol, the unreacted 3,3,5-trimethylcyclohexanone, and the by-products obtained in step (d) towards waste recovery and waste removal.

2. The process of claim 1, wherein the process is conducted continuously.

3. The process of claim 1, wherein in step (c) gaseous acidic catalyst and water are removed from the reaction mixture by distillation.

4. The process of claim 1, wherein in step (d) the larger amount of the obtained 3,3,5-trimethylcyclohexylidene bisphenol is separated from the unreacted phenol, from the unreacted 3,3,5-trimethylcyclohexanone, and from the by-products by at least the following steps:
(d1) converting the obtained 3,3,5-trimethylcyclohexylidene bisphenol into a 3,3,5-trimethylcyclohexylidene-bisphenol-phenol-adduct, and
(d2) separating the 3,3,5-trimethylcyclohexylidene-bisphenol-phenol-adduct by crystallization and filtration.

5. The process of claim 1, wherein in a step (d3) the obtained 3,3,5-trimethylcyclohexylidene-bisphenol-phenol-adduct is purified by recrystallization in phenol towards at least 99.9 wt.-% purity.

6. The process of claim 1, wherein in a step (d4) 3,3,5-trimethylcyclohexylidene bisphenol is obtained from the separated 3,3,5-trimethylcyclohexylidene-bisphenol-phenol-adduct by the removal of phenol from the 3,3,5-trimethylcyclohexylidene-bisphenol-phenol-adduct.

7. The process of claim 1, wherein the initial amount of 3,3,5-trimethylcyclohexanone comprises an amount of freshly added 3,3,5-trimethylcyclohexanone and an amount of unreacted 3,3,5-trimethylcyclohexanone and wherein the molar ratio between the amount of freshly added 3,3,5-trimethylcyclohexanone and the amount of unreacted 3,3,5-trimethylcyclohexanone is from 2:° 1 to 15:1.

8. The process of claim 1, wherein the initial amount of phenol comprises an amount of freshly added phenol and an amount of unreacted phenol and wherein the molar ratio between the amount of freshly added phenol and the amount of unreacted phenol is less than 1:3.

9. The process of claim 1, wherein in step (a) the initial mixture comprises 5 to 25 wt.-% by-products, or from 1 to 4 wt.-% of by-products.

10. The process of claim 1, wherein no water is added.

11. The process of claim 1, wherein the reaction between 3,3,5-trimethylcyclohexanone and phenol is conducted in a reaction vessel.

12. The process of claim 1, wherein the reaction temperature in the reaction vessel is at least 30° C. and at most 40° C.

13. The process of claim 1, wherein the 3,3,5-trimethylcyclohexylidene bisphenol is obtained with a purity of greater 95 wt. %.

14. The process of claim 1, wherein in step (b) the obtained reaction mixture comprises 55 to 70 wt.-% phenol, less than 5 wt. % 3,3,5-trimethylcyclohexanone, from 15 to 22 wt.-% 3,3,5-trimethylcyclohexylidene-bisphenol, from 3.5 to 5.5 wt.-% gaseous acidic catalyst, from 0.5 to 2 wt.-% water, and 5 to 20 wt.-% of by-products, wherein the sum of the amounts of unreacted phenol, unreacted 3,3,5-trimethylcyclohexanone, 3,3,5-trimethylcyclohexylidene bisphenol, water, and by-products is 100 wt.-%.

15. The process of claim 1, wherein
in step (d) the larger amount of the obtained 3,3,5-trimethylcyclohexylidene bisphenol is an amount in a range of 70 to 95 wt-%;
in step (e1) the larger amount of the remaining reaction mixture of (d) containing 3,3,5-trimethylcyclohexylidene bisphenol, the unreacted phenol, the unreacted 3,3,5-trimethylcyclohexanone, and the by-products obtained in step (d) is an amount in a range of 90 to 99.9 wt.-%;
in step (f) the smaller amount of the remaining reaction mixture of (d) containing 3,3,5-trimethylcyclohexylidene bisphenol, the unreacted phenol, the unreacted 3,3,5-trimethylcyclohexanone, and the by-products obtained in step (d) is an amount in the range of 10% to 0.1 wt.-%;
in step (e2) the smaller amount of the obtained 3,3,5-trimethylcyclohexylidene bisphenol, the unreacted phenol, the unreacted 3,3,5-trimethylcyclohexanone, and the by-products obtained in step (d) is an amount in a range of 50 to 90 wt.-%; and
in step (e3) the smaller amount of the obtained 3,3,5-trimethylcyclohexylidene bisphenol, the unreacted phenol, the unreacted 3,3,5-trimethylcyclohexanone, and the by-products obtained in step (d) is an amount in a range of 50 to 90 wt.-%.

16. The process of claim 1, wherein
in step (d) the larger amount of the obtained 3,3,5-trimethylcyclohexylidene bisphenol is an amount in a range of 80 to 90 wt-%;
in step (e1) the larger amount of the remaining reaction mixture of (d) containing 3,3,5-trimethylcyclohexylidene bisphenol, the unreacted phenol, the unreacted 3,3,5-trimethylcyclohexanone, and the by-products obtained in step (d) is an amount in a range of 95 to 99 wt.-%;
in step (f) the smaller amount of the remaining reaction mixture of (d) containing 3,3,5-trimethylcyclohexylidene bisphenol, the unreacted phenol, the unreacted 3,3,5-trimethylcyclohexanone, and the by-products obtained in step (d) is an amount in the range of 5% to 1 wt.-%;
in step (e2) the smaller amount of the obtained 3,3,5-trimethylcyclohexylidene bisphenol, the unreacted phenol, the unreacted 3,3,5-trimethylcyclohexanone, and the by-products obtained in step (d) is an amount in a range of 60 to 80 wt.-%; and
in step (e3) the smaller amount of the obtained 3,3,5-trimethylcyclohexylidene bisphenol, the unreacted phenol, the unreacted 3,3,5-trimethylcyclohexanone, and the by-products obtained in step (d) is an amount in a range of 60 to 80 wt.-%.

17. The process of claim 3, wherein the distillation uses a distillation column having a bottom and the bottom temperature being 130° C. at most.

18. The process of claim 6, wherein in the step (d4), the 3,3,5-trimethylcyclohexylidene bisphenol is obtained from the separated 3,3,5-trimethylcyclohexylidene-bisphenol-phenol-adduct by the removal of phenol from the 3,3,5-trimethylcyclohexylidene-bisphenol-phenol-adduct by drying.

19. The process of claim 7, wherein the amount of unreacted 3,3,5-trimethylcyclohexanone is from 5:1 to 10:1.

20. The process of claim 8, wherein the molar ratio between the amount of freshly added phenol and the amount of unreacted phenol is in a range of 1:7 to 1:4.
